(19) United States
(12) Patent Application Publication
(10) Pub. No.: US 2018/0190370 A1
(43) Pub. Date: Jul. 5, 2018

(54) Universal Medical Access Card System and Process Thereof

(71) Applicant: Robert C. VILLARE, Woodbury, NJ (US)

(72) Inventor: Robert C. VILLARE, Woodbury, NJ (US)

(21) Appl. No.: 15/900,138

(22) Filed: Feb. 20, 2018

Related U.S. Application Data
(63) Continuation of application No. 14/725,503, filed on May 29, 2015, now abandoned.
(60) Provisional application No. 62/017,283, filed on Jun. 26, 2014.

(51) Int. Cl. G16H 10/60; G16H 10/65

(52) U.S. Cl. CPC G16H 10/60; G16H 10/65; G06F 19/322

(57) ABSTRACT

A universal medical access card (UMAC) system that allows patients to conveniently and securely maintain and possess their own medical records. The UMAC system includes a portable UMAC device, a plurality of first-level service providers, a plurality of second-level service providers and a UMAC database. The UMAC device securely downloads and stores medical records of a patient in memory and can be updated by one or more of the first-level service providers, one or more of the second-level service providers and/or the UMAC database. Also, the UMAC database can be updated by one or more of the first-level service providers, one or more of the second-level service providers and/or the UMAC device.

### CROSS-REFERENCE TO RELATED APPLICATIONS

This Continuation application claims the benefit under 35 U.S.C. § 120 of application Ser. No. 14/725,503 filed on May 29, 2015, which in turn claims the benefit under 35 U.S.C. § 119(e) of Application Ser. No. 62/017,283 filed on Jun. 26, 2014 and both of which are entitled “Universal Medical Access Card System and Process Thereof”, and both of which are incorporated by reference in their entireties.

### FIELD OF THE INVENTION

The present invention relates to a universal medical access system, and in particular to a universal medical access system that provides a patient with a portable universal medical access device that contains their medical records.

### BACKGROUND OF THE INVENTION

The need and desire to create and maintain accurate medical records is known. In addition, frustration related to patients not having convenient access to their medical records is also known. As such, numerous consultants and IT specialists have tried to merge existing hospital and physician systems with little success.

Prior art systems typically use or have a plurality of different locations that have a portion of a patient's records. However, a single and convenient location, device, etc. that maintains up to date and complete records for a given patient, and which is totally in the possession of the patient is not presently available. Therefore, a system, card, etc. that provides complete and up-to-date medical records for a patient and which is in their possession at any given time would be desirable and fulfill an unmet medical need.

### SUMMARY OF THE INVENTION

A universal medical access card (UMAC) system that allows patients to conveniently and securely maintain and possess their own medical records is provided. The UMAC system includes a portable UMAC device that has a micro-controller, memory, and a software module with a UMAC system software program. The microcontroller is operable to execute the UMAC system software program and the UMAC system software program securely downloads and stores medical records of a patient in the memory. The UMAC system software program is also operable to securely display the medical records on a display device and securely provide the medical records to a first-level service provider.

The UMAC system also includes a plurality of second-level service providers with each of the second-level service providers having a second-level UMAC device reader that can securely read at least a portion of the medical records from the UMAC device. In addition, each of the second-level service providers can execute a request associated with the at least a portion of the medical records and electronically store executed request data.

The UMAC system further includes a UMAC central records database that can securely read and store the executed request data from each of the second-level service providers. The UMAC central records database can provide the executed request data to the UMAC device, the UMAC system software program can read the executed request data from the UMAC records database, store the executed request data in the UMAC device memory, and thereby obtain up-to-date medical records on a patient that has possession and/or owns the portable UMAC device.

In some instances, the portable UMAC device is owned by a patient and thus is a portable patient-owned UMAC device that contains the medical records of the patient. As such, the patient has in their possession the portable UMAC device which can include, but is not limited to, a credit card type device, a USB flash drive device, and the like that contains their own medical records and information. Stated differently, the UMAC device can be a swipe card, a memory stick, a memory chip and the like.

The first-level service provider can be a UMAC system approved service provider that performs an authorized medical examination on a patient that has or owns a portable UMAC device. Such UMAC system approved service providers can include physicians, hospitals, emergency care facilities, urgent care facilities, etc. In some instances, the UMAC system approved service providers can also include dentists, chiropractors, massage therapists, laser treatment therapists, etc. Such UMAC system approved service providers have a first-level UMAC device reader that can securely read from and write to the portable UMAC device.

The second-level UMAC service providers are providers that execute a request and/or provide a service to or for a patient without an authorized medical examination performed thereby. Such second-level providers include medical service providers such as pharmacists; pharmacies; and medical testing laboratories such as a blood testing laboratory, an x-ray laboratory, an MRI laboratory, an ultrasound laboratory, an endoscopic laboratory, a neurodiagnostic testing laboratory, a cardiac testing laboratory and the like. The second-level service providers have a second-level UMAC device reader that can only read from the portable UMAC device.

For example and for illustrative purposes only, the second-level UMAC device can read a prescription that has been given to a patient and recorded on the device. Such prescriptions can include pharmaceutical prescriptions, requests for blood testing, x-rays, an MRI, an ultrasound, an endoscopic examination, neurodiagnostic examination, a cardiac examination, etc. As such, the second-level UMAC device reader can read requests for the filling of a pharmaceutical prescription, testing, and the like, but cannot write any results onto the device.

In the alternative, a second-level UMAC service provider can be a UMAC system approved service provider that has a first-level UMAC device reader which allows or affords for executed request data to be written onto the UMAC device. For example and for illustrative purposes only, a particular pharmacy or chain of pharmacies can be a UMAC system approved service provider and thus have a first-level UMAC device reader that affords for writing to the UMAC device that a particular prescription has indeed been filled and provided to the patient.

In some instances, and when a second-level service provider is not a UMAC system approved service provider, the second-level service provider transmits executed request and/or service data to the UMAC records database. The UMAC records database can then subsequently update the UMAC device the next time the patient logs onto the UMAC system. It is appreciated that a second-level UMAC system approved service provider with a first-level UMAC device reader can also transmit executed request and/or service data to the UMAC records database, which in turn affords for updating the UMAC device the next time the patient logs onto the UMAC system.

In at least one embodiment, the first-level UMAC device reader and/or the second-level UMAC device reader securely reads and/or writes at least a portion of the medical records data from and/or to the UMAC device via a physical connection therewith. In at least one other embodiment, the first-level UMAC device reader and/or the second-level UMAC device reader securely reads and/or writes at least a portion of the medical records data from or to the UMAC device via a wireless connection therewith. In still another embodiment, the first-level UMAC device reader and/or the second-level UMAC device reader securely reads and/or writes at least a portion of the medical records data from and/or to the UMAC device via a physical connection and a wireless connection therewith.

It is appreciated that the UMAC central records database can securely read and store the executed request data from each of the second-level service providers. In some instances, the reading of the executed request data occurs via the internet. In addition, the UMAC central records database provides the executed request data to the UMAC device via a secured internet connection.

### BRIEF DESCRIPTION OF THE DRAWINGS

FIG. 1 is a schematic illustration of a universal medical access (UMA) system according to an embodiment disclosed herein;

FIG. 2 is a schematic illustration of another embodiment of a UMAC system according to an embodiment disclosed herein;

FIG. 3 is a schematic illustration of yet another embodiment of a UMAC system disclosed herein;

FIG. 4 is a schematic flowchart illustrating operation of a UMAC system according to an embodiment disclosed herein;

FIG. 5 is a schematic illustration of a UMAC device according to an embodiment disclosed herein;

FIG. 6 is a schematic illustration illustrating operation of a UMAC system software program according to an embodiment disclosed herein;

FIG. 7 is a schematic illustration of a UMAC system according to an embodiment disclosed herein;

FIG. 8 is a schematic illustration for a patient using the UMAC system according to an embodiment disclosed herein; and

FIG. 9 is a schematic illustration of the operation of a central processing unit for a UMAC device according to an embodiment disclosed herein.

### DETAILED DESCRIPTION OF THE INVENTION

The present invention provides a universal medical access card (UMAC) system and process for keeping, inputting, accessing, storing, delivering, and aggregating health records for a patient. The UMAC system provides a personal, patient-centric key to access and updating of the patient's information. The UMAC device system also serves as a means to back up records in the event of a catastrophe and can be used to enhance homeland security.

The UMAC system includes a personal UMAC device that stores all medical-related information for a particular patient. Stated differently, each patient has a UMAC device with all of their medical information, records, etc. thereon. The UMAC device can be any electronic storage device known to those skilled in the art, illustratively including a USB flash drive, a credit card-type storage device, and the like. The UMAC device has sufficient memory/storage that is capable to hold and store the medical information/records for a given patient. Such storage can include up to 2 gigabytes of storage, or in the alternative, up to 4 gigabytes of storage, or in another alternative, up to 10 gigabytes of storage.

In some instances, the UMAC device is a smart card embedded with an integrated circuit that can encrypt and securely store patient information. The UMAC smart card allows access to the patient information only to authorized users, e.g. the patient and approved healthcare providers. In addition, the UMAC smart card can be accessed via a con-tactless and/or a contact protocol. It is appreciated that the integrated circuit of the UMAC smart card can include a microprocessor, memory, and the like.

The UMAC device also has a software application thereon, e.g. an object code enabled smart card app that interfaces with a web browser via a web-servlet. It is appreciated that the software application can be in any programming language known to those skilled in the art, illustratively including JavaScript, C++, Perl, Python, Visual Basic and the like. The app allows for the UMAC device to be automatically uploaded into a standard web browser such that active browsing and connection to a specific patient's master medical record file is provided. As such, access to the patient's master medical record file can be obtained through any computerized device known to those skilled in the art such as a desktop computer, a laptop computer, a personal electronic device (PED) such as an iPad, a personal tablet, a smartphone, and the like.

The app on the UMAC device can also check for internet connectivity and if available, redirect an individual computerized device to a UMAC web site and/or UMAC web-site login page. The individual can log into the UMAC web-site and the UMAC device can be automatically updated before displaying health records. The system/process includes a web-based mobile electronic medical information, health safety system, device/tool, and application (app) for tracking, obtaining, storing, and aggregating patient-centric information. The system/process also reengineers the paradigm of input, storage, and retrieval of medical records and thereby aggregates a patient's records from various sources of contact in the healthcare system and enables the patient to own, carry, and access his/her own records.

The UMAC website can have a UMAC database that receives medical information from diverse patient service providers and the patient. Such service providers include but are not limited to doctors, physical therapy facilities, testing facilities, pharmaceutical facilities, hospitals, emergency care units, etc. Stated differently, service providers for the patient can transmit information on services provided to the patient to the UMAC database, and the UMAC database can in turn update the patient's UMAC device. In this manner, a unique paradigm in which a patient-centric process and system architecture is provided in which a patient has in their possession a simple and convenient device that contains all of their medical records and can be easily accessed whenever desired.

Turning now to FIG. 1, an embodiment of a UMAC system is shown generally at reference numeral 10. The UMAC system 10 includes a UMAC device, e.g. an electronic device in the form of a smart card, USB flash drive, etc., at reference numeral 100. It is appreciated that the UMAC device 100 is associated with a particular patient. In addition, medical-related services are present at service provider 110 and a UMAC database at 120. It is also appreciated that the term UMAC database includes or refers to other components that are part of a digital medical records storage facility such as a server with memory, an electronic control unit (ECU), and the like. As shown in the figure, the UMAC device 100 is in communication with the services 110 which in turn is in communication with the UMAC database 120. Finally, the UMAC database 120 is in communication with the UMAC device 100.

The communication illustrated in FIG. 1 is a “one-way communication” from the UMAC device 100 to the service provider 110, from the service provider 110 to the UMAC database 120, and from the UMAC database 120 to the UMAC device 100. As such, the service provider 110 cannot upload information to the UMAC device 100, i.e. only the UMAC database 120 can upload and update information to the UMAC device 100.

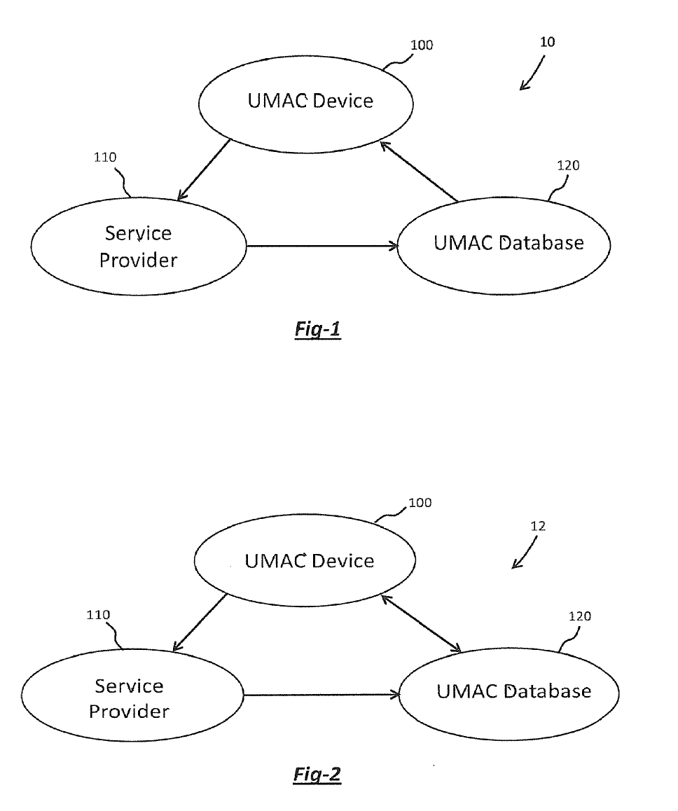

Another embodiment of the system is shown in FIG. 2 at reference numeral 12. The embodiment 12 is similar to the embodiment 10 except that the UMAC device 100 and UMAC database 120 have two-way communication as illustrated by the double-headed arrow between the patient with UMAC device 100 and UMAC database 120. In this manner, the UMAC database 120 can be used to update the UMAC device 100 and the UMAC device 100 can be used to update the UMAC database 120. Such updating of the UMAC database 120 by the UMAC device 100 allows for retrieval of information in the event of a catastrophic event with respect to the UMAC database 120, e.g. in the event the UMAC database 120 is destroyed by an act of God, terrorism, etc.

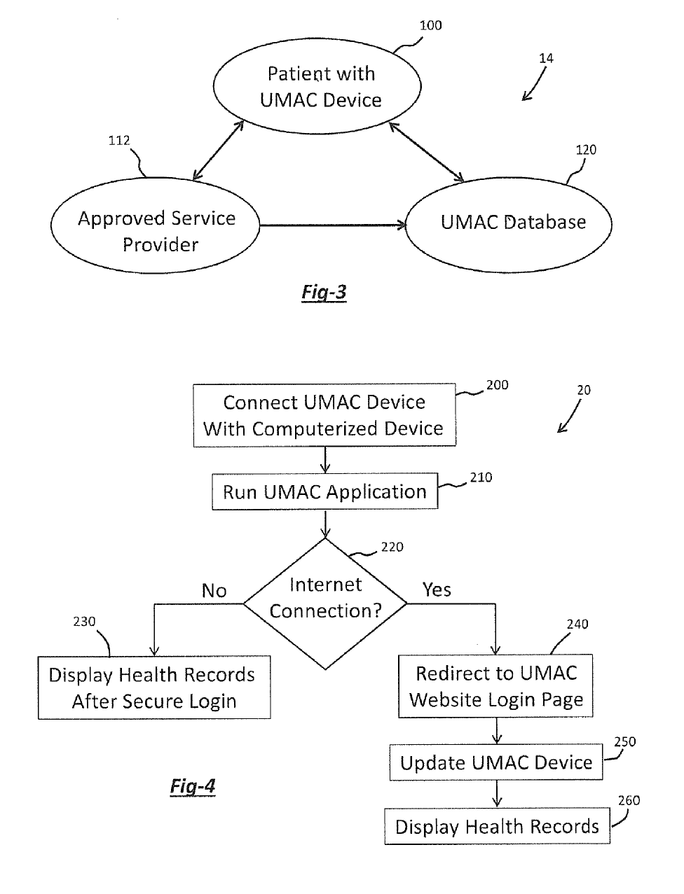

Yet another embodiment of the UMAC system is shown in FIG. 3 at reference numeral 14. The embodiment 14 includes two-way communication between the UMAC device 100 and approved service providers 112. In this manner, services are provided to the patient by an approved UMAC system provider 112 which can update the patient's UMAC device 100.

Referring now to FIG. 4, a flowchart illustrating an embodiment of the UMAC system in operation is shown generally at reference numeral 20. The process or flowchart 20 starts with an initial step 200 in which a UMAC device is connected with a computerized device. Once connected to the computerized device, e.g. wirelessly and/or through a physical port using Near Field Communication (NFC) technology, a UMAC software application is initiated and run or executed at step 210. Upon initialization of the UMAC application (app), the app checks for an internet connection at step 220. If an internet connection is not available, then the app displays the health records on the computerized device using any standard web browser known to those skilled in the art after a secure login at step 230.

The health records can include information on hospital stays, medications having been taken in the past and/or medications currently being taken, tests that have been prescribed for the patient, results of any tests that have been prescribed and executed or performed on the patient, physical therapy prescribed for the patient and the results of physical therapy participated in by the patient, a list of doctors having been seen by the patient and/or currently being seen by the patient, allergy information for the patient, surgeries performed on the patient, vaccination information for the patient, and the like.

In the event that an internet connection is available at step 220, the UMAC app redirects the computerized device to a UMAC web site login page at step 240. The individual can then log into the UMAC website and the patient's UMAC device can be updated at step 250. It is appreciated that the UMAC device is updated with recent information/data obtained by service providers that have provided services, information, etc. to/for the patient. After and/or during updating of the UMAC device, the patient's health records are displayed at step 260.

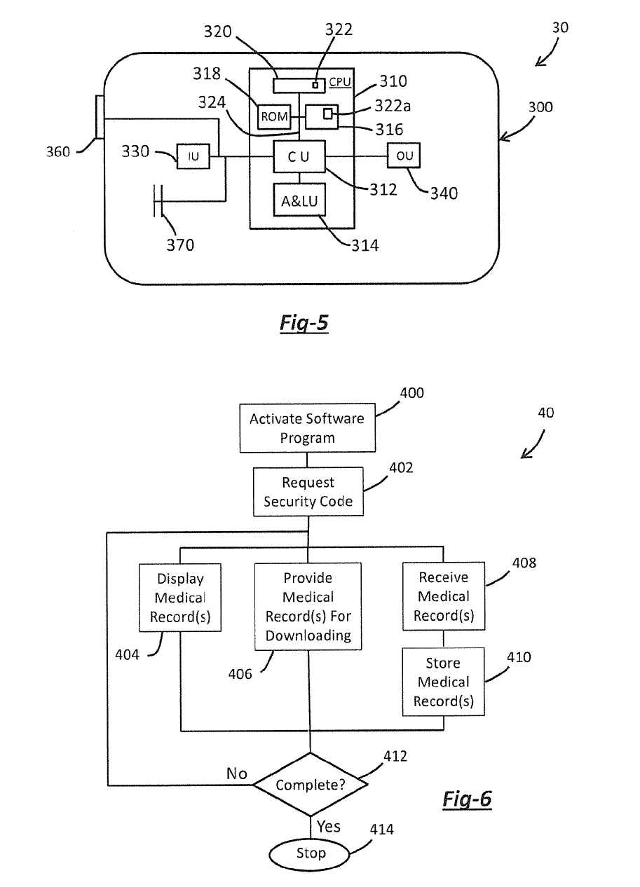

Turning now to FIG. 5, a schematic illustration of a UMAC device is shown generally at reference numeral 30. The device 30 includes a housing 300 with a central processing unit (CPU) 310. The CPU 310 includes a control unit 312 and an arithmetic and logic unit 314. The CPU 310 also includes memory, such as ready access memory (RAM) 316 and read-only memory (ROM) 318. Additional storage 320 can also be present. A software module 322 and/or 322a can be located on the storage 320 and/or RAM 316. As is known to those skilled in the art, a software module can be permanently stored on storage 320 and then when initiated have a portion thereof stored on RAM 316. It is also appreciated that the controller unit 312 is in communication with the various other components of the CPU via one or more buses 324.

The UMAC device 30 also includes an input unit 330 and an output unit 340. Optionally, a physical connection port or plug 360 can be included and be in electrical connection with the input unit 330, as can be a wireless receiver and/or transmitter 370. It is appreciated that the UMAC device can have an NFC tag, use NFC technology and the like.

FIG. 6 schematically illustrates operation of the UMAC device 30 with the CPU 310 at reference numeral 40. In operation, the controller unit 312 activates a software program located within the software module 322 at step 400. The software program can request a security code at step 402 and upon entry of a correct code, password, etc., can display medical records located on the RAM 316 and/or the storage 320 at step 404. In addition, the medical records can be provided for downloading at step 406. The controller unit 312 in combination with the software program also affords for receiving medical records at step 408 and storing the medical records on the RAM 316 and/or storage 320 at step 410.

At step 412, a decision tree is present and if a patient has completed a review of their medical records and/or completed receiving medical records and having them stored on the UMAC device 30, the decision tree 412 goes to stop at step 414. In the alternative, if the patient has not completed their desired review of the medical records, providing the medical records for downloading, and/or receiving and storing medical records, the decision tree redirects the system back to the options 404, 406, 408, 410 discussed above.

FIG. 7 provides a schematic overview of a UMAC system at reference numeral 50. The UMAC system includes a UMAC device 100 and a plurality of first-level service providers 512, 514, . . . 520, and a plurality of second-level service providers 532, 534, . . . 540. As shown in the figure, each of the first-level service providers 512, 514, . . . 520 can have a first-level UMAC device reader 513, 515, . . . 521, and each of the second-level service providers can have a second-level UMAC device reader 533, 535, . . . 541. It is appreciated that when a patient visits a first-level service provider and/or a second-level service provider, their UMAC device 100 can be placed into communication with the respective first-level UMAC device reader and/or second-level UMAC device reader. In this manner, medical records, prescriptions, testing data requests, executed testing data, and the like can be transferred between the UMAC device 100 and one or more first-level service providers 512, 514, . . . 520 and/or one or more second-level service providers 532, 534, . . . 540.

Also shown in FIG. 7 is a central hub or UMAC database system 550 that can be in communication with the UMAC device 100, of the first-level service providers 512, 514, . . . 520, and each of the second-level service providers 532, 534, . . . 540 via internet connection 560. In this manner, the UMAC database 550 can be updated with data from the UMAC device 100, one or more first-level service providers 512, 514, . . . 520 and/or one more second-level service providers 532, 534, . . . 540. Furthermore, the UMAC device 100 can be updated with data from one or more first-level service providers 512, 514, . . . 520, one more second-level service providers 532, 534, . . . 540 and/or the UMAC database 550. As such, the UMAC system 50 affords for the UMAC device 100 to contain up-to-date medical records for the patient.

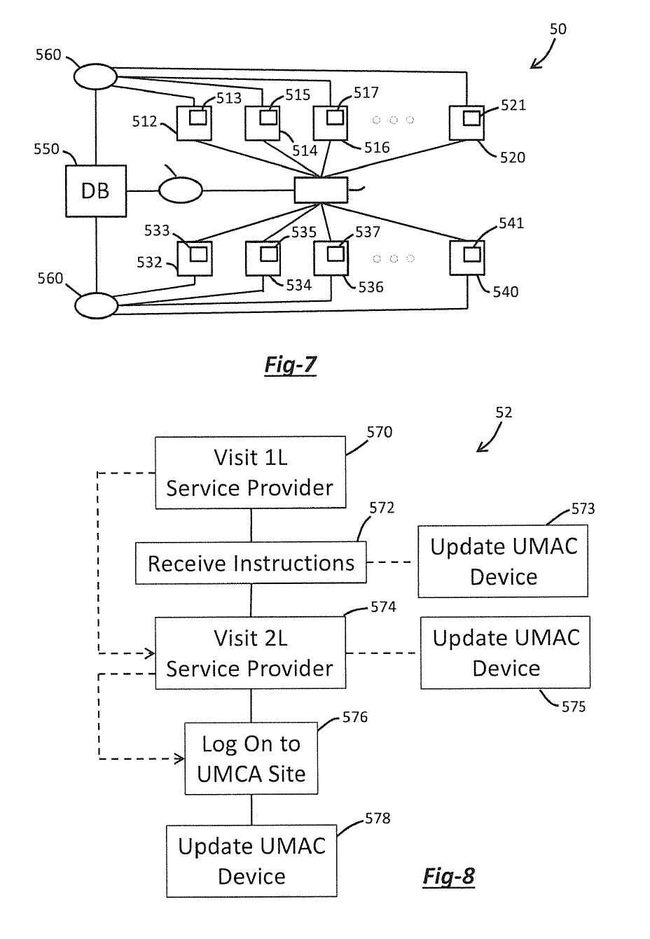

Turning now to FIG. 8, a typical use or operation of a UMAC device by a patient is shown generally at reference numeral 52. For example and for illustrative purposes only, a patient visits a first-level (1L) service provider at step 570. As stated above, the first-level service provider can be an approved UMAC service provider such as a physician, a hospital, an emergency care unit, an urgent care unit, etc. Upon visiting the first-level service provider, the patient receives instructions at step 572. The instructions include any instructions received from such a service provider such as instructions to have a prescription filled, to have a certain testing procedure completed, and the like. The patient's UMAC device can optionally be updated at step 573. For example, the UMAC device can be updated to include a pharmaceutical prescription from a physician, a prescription for an MRI, and the like. In the alternative, the first-level service provider can send such a prescription or instructions to a second-level (2L) service provider without updating the UMAC device of the patient. In another alternative, the first level-service provider can update the UMAC device at step 573 and send a prescription or instructions to a second-level service provider.

The patient then visits a particular and desired second-level service provider at 574 and the second-level service provider executes a request from the first-level service provider. For example and for illustrative purposes only, the second-level service provider can fill a pharmaceutical prescription for the patient, perform an MRI on the patient, and the like. The UMAC device can optionally be updated by and/or at the second-level service provider location, e.g. indicating that the instruction, request, etc. has been executed or completed. In the alternative, the second-level service provider cannot update the UMAC device but instead transmits the executed request data to the central UMAC database.

The patient can then log onto the UMAC site, e.g. using a computer and the internet, at step 576. Upon logging onto the UMAC site, the patient's UMAC device can be updated at step 578. In this manner, a patient can maintain and have in their possession their complete and up-to-date medical records.

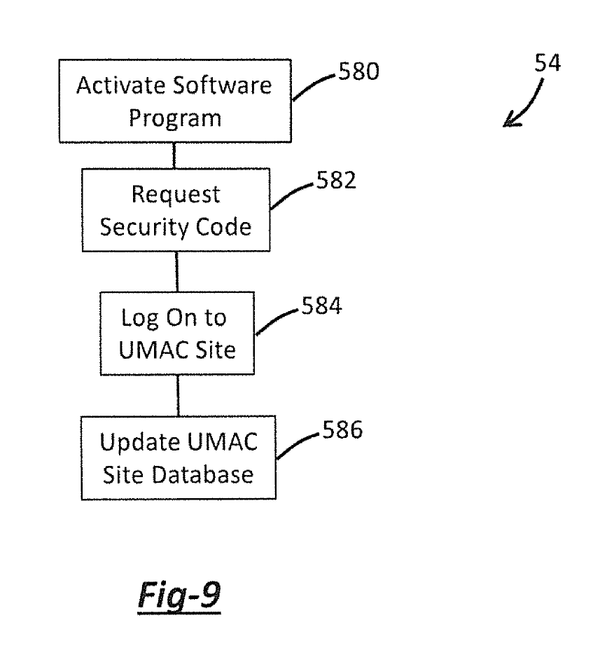

The UMAC system can also be used to rebuild or restore the central UMAC database. For example, in the event of an act of God, a terrorist attack, etc. that results in the central UMAC database being destroyed, patients can log into the UMAC site and update the UMAC database with all of their medical records. For example and for illustrative purposes only, FIG. 9 provides such a schematic illustration at reference numeral 54. In particular, the patient can use a computer, a first-level UMAC device reader, and/or a second-level UMAC device reader, which in turn would activate the software program of the UMAC device 100 at step 580. As stated above, the software program would request a security code at step 582, and upon entry of a correct password, security code, etc., log on to the UMAC site at step 584. Thereafter, the UMAC database could read and download the medical records for the patient at step 586. In this manner, the UMAC system provides not only for a patient to have in their possession complete and up-to-date medical records, but also allows the UMAC system to be rebuilt and/or restored by the individual patients.

In operation, the UMAC system has over 1,000 patients with their own UMAC device, preferably more than 10,000 patients with their own UMAC device, and more preferably more than 100,000 patients with their own UMAC device. In some instances, the UMAC system has more than 500,000 patients with their own UMAC device and can have more than 1,000,000 patients with their own UMAC device. In addition, the UMAC system includes at least 100 first-level service providers, preferably more than 1,000 first-level service providers, more preferably more than 10,000 first-level service providers, and still more preferably more than 100, 000 first-level service providers, each of the first-level service providers having a first-level UMAC device reader.

The UMAC system also has a plurality of second-level service providers, preferably more than 1,000 second-level service providers, more preferably more than 10,000 second-level service providers, and even more preferably more than 100,000 second-level service providers, each of the second-level service providers having a second-level UMAC device reader.

The UMAC database can be a centrally located database located on one or more computer servers, or in the alternative located in the “cloud”. In the alternative, the UMAC database can be located at different locations around the country and the world but be in communication with each other such that a unified UMAC system/database is provided.

The display of health records can be in the form of a user-friendly page with tabs that direct the patient or a service provider to major categories related to the medical information. For example, a critical health summary page can be readily available or selected by an individual and have links to view various tests, test results, and the like such as MRI reports, x-rays, allergy information, current medications, etc.

It should be appreciated that the UMAC device is owned and maintained by the patient and thus the patient is empowered with ownership of their own medical records. It is also appreciated that the UMAC devices, the UMAC web site, and the like have desired security measures that meet the HIPAA security rule and allow a patient to only have access to their UMAC device and UMAC database account. Such security measures naturally include passwords, biometric identification, and the like.

The UMAC device and/or UMAC database can be accessed only by members of the system such as patients, doctors, service providers, and the like. The UMAC database can have a translation module or function that can translate medical information into one or more languages with the UMAC device then updated with the translated medical information. In addition, the UMAC device and/or the app on the card affords for searching of the medical information on the card, printing of the information, etc. As such, the UMAC device system can replace the medical “clipboard” used by doctors and hospitals, doctors' offices, etc.

In order to better illustrate the invention but not limit its scope in any manner, one or more examples of the use of the UMAC device and the UMAC system are discussed below.

A patient with a UMAC device in the form of a UMAC device that fits within the patient's wallet schedules a visit with their internal medicine doctor. During scheduling of the doctor's visit, either online or by a phone call, the doctor's office obtains a unique identification number/code of the patient's UMAC device. The date and time of the upcoming visit is entered into the doctor's computerized patient scheduling system and then transmitted to the UMAC database. The UMAC database then updates the patient's UMAC device during the next login of the patient with the UMAC database.

Thereafter, the patient visits the doctor and as a result of the doctor's visit, blood work is prescribed for the patient, as is a chest x-ray and one behind-the-counter prescription and one over-the-counter prescription. Again, the prescriptions for the blood work, x-ray, and medications are entered into the doctor's computerized system and then trans-mitted to the UMAC database. The UMAC database then updates the patient's UMAC device during the next login of the patient with the UMAC system.

It is appreciated that during the doctor's visit, the doctor and/or one of his/her representatives can connect the patient's UMAC device to a computerized device. The UMAC device having the app described above initiates and executes viewing of the patient's medical records. As such, the doctor has instant access to the patient's medical records, test results, and the like that have been performed and/or obtained from past activity.

The patient subsequently has their blood drawn and a chest x-ray taken. Also, the patient goes to their local pharmacy and purchases the one over-the-counter and one behind-the-counter medication. During the taking of the patient's blood, the patient's UMAC device is scanned and the fact that blood has been drawn from the patient is communicated by the service provider to the UMAC database. In addition, the fact that an actual chest x-ray of the patient has been obtained is communicated by the x-ray service provider to the UMAC database. Finally, purchase of the medications at the patient's local pharmacy is also communicated to the UMAC database. As stated above, such information now in the UMAC database is used to update the patient's UMAC device during the next login by the patient with the UMAC system.

Upon obtaining results from the blood work, the blood testing lab/service provider communicates such results with the UMAC database. In addition, upon analysis of the chest x-ray and production of a chest x-ray report by an x-ray specialist/technician/doctor, the x-ray report is communicated to the UMAC database. Such information is also used to update the patient's UMAC device.

Given the above, the next time the patient visits a healthcare provider, the above-discussed medical tests, results, etc. are easily accessed thereby. In addition, the medical records are in the possession of the patient and provide a new sense of ownership by the patient.

The inventive system/process provides a system that creates a national membership enabling true coordination, communication, and collaboration of care. As such, the dimensions of geography, distance, and time no longer present barriers to patients and/or healthcare providers. In addition, patient members benefit from elimination of redundancy, fraud, and abuse, and may gain incentive from insurers who offer reduced premiums to members of the national network. Insurers can also offer incentive payments to physician members who benefit from instant access to all specialty recommendations for diagnosis and treatment.

The system/process also includes alerts and reminders to empower patients to have greater control and participation in their own healthcare. Furthermore, peer review occurs via consultations with specialists who have instant access to a complete, aggregated medical record for a patient, thereby allowing delivery of best quality care while keeping costs down. It is appreciated that insurers and government benefits from reduced costs.

In addition to the above, the need to provide special reports to the government becomes a relic and reduces government expense and burden of analysis on provider delivery of patient-centric care. Stated differently, the process/system enables patients working with private healthcare to accomplish this task. Also, the costs and time spent in malpractice are potentially reduced dramatically for users of the system since expert opinions may be rendered via expert insurance panels of medical professionals or medical society organized panels with member providers participating in this collaboration.

The above examples, embodiments, etc. are provided for illustrative purposes only and are not meant to limit the invention in any way. Changes, modifications, etc. can be made by those skilled in the art and yet still fall within the scope of the invention. As such, it is the claims and all equivalents thereof that define the scope of the invention.

I claim:

1. A universal medical access (UMA) system comprising:
a portable universal medical access card (UMAC) device, said UMAC device having a microcontroller, memory and a software module with a UMA system software program, said microcontroller operable to execute said UMA system software program and said UMA system software program operable to download and store medical records of a patient in said memory, said UMA system software program also operable to display said medical records on a display device and provide said medical records to a first-level service provider;
a plurality of second-level service providers, each of said plurality of second-level service providers having a second-level UMAC device reader operable to read at least a portion of said medical records from said UMAC device, each of said plurality of second-level service providers operable to execute a request associated with said at least a portion of said medical records and store executed request data;
a UMAC central records database operable to read and store said executed request data from each of said plurality of second-level service providers, said UMAC central records database also operable to provide said executed request data to said UMAC device; and
wherein said UMA system software program is operable to read said executed request data from said UMAC records database and store said executed request data in said memory and maintain up-to-date medical records on the patient on said patient-owned UMAC device.

2. The UMA system of claim 1, wherein said portable UMAC device is a portable patient-owned UMAC device containing medical records for a patient that owns the UMAC device.

3. The UMA system of claim 1, wherein said first-level service provider has a first-level UMAC device reader, said first-level UMAC device reader operable to read from and write to said portable UMAC device.

4. The UMA system of claim 3, wherein said second-level UMAC device reader is operable to only read from said portable UMAC device.

5. The UMA system of claim 4, wherein said first-level service provider is a UMA system approved service provider that performs an authorized medical examination of a patient that has said portable UMAC device.

6. The UMA system of claim 5, wherein said UMA system approved service provider is at least one of a physician, a hospital, an emergency care facility and an urgent care facility, a dentist and a chiropractor.

7. The UMA system of claim 6, wherein said second-level service provider is a service provider that provides a service to a patient without an authorized medical examination that has said portable UMAC device.

8. The UMA system of claim 7, wherein said second-level service provider is at least one of a pharmacy and a medical testing laboratory.

9. The UMA system of claim 8, wherein said medical testing laboratory is at least one of a blood testing laboratory, an x-ray laboratory, a magnetic resonance imaging (MRI) laboratory, an ultrasound laboratory, an endoscopic laboratory, a neurodiagnostic testing laboratory and a cardiac testing laboratory.

10. The UMA system of claim 1, wherein second-level UMAC device reader reads at least a portion of said medical records data from said UMAC device via a physical connection with said UMAC device.

11. The UMA system of claim 1, wherein said second-level UMAC device reader reads at least a portion of said medical records data from said UMAC device via a wireless connection with said UMAC device.

12. The UMA system of claim 11, wherein said UMAC device has a near field communication (NFC) tag.

13. The UMA system of claim 1, wherein said second-level UMAC device reader reads at least a portion of said medical records data from said UMAC device via a physical connection and a wireless connection with said UMAC device.

14. The UMA system of claim 1, wherein said UMAC central records database reads and stores said executed request data from each of said plurality of service providers via a secured internet connection.

15. The UMA system of claim 1, wherein said UMAC central records database provides said executed request data to said UMAC device via a secured internet connection.
